(19) United States
(12) Patent Application Publication
(10) Pub. No.: US 2006/0166271 A1
(43) Pub. Date: Jul. 27, 2006

(54) Non-radioactive schilling test

(76) Inventors: Lars Erik Berglund, Odder (DK); Ebba Nexoe, Asbyhoej (DK)

Correspondence Address: GIFFORD, KRASS, GROH, SPRINKLE & CITKOWSKI, P.C, PO BOX 7021, TROY, MI, 48007-7021, US

(21) Appl. No.: 10/549,077

(22) PCT Filed: Mar. 12, 2004
(86) PCT No.: PCT/DK04/00163

§ 371 Date: Dec. 30, 2005

(30) Foreign Application Priority Data
Mar. 14, 2003 (GB) 03059045

(51) Int. Cl. G01N 33/53

(52) U.S. Cl. 435/7.1

(57) ABSTRACT

The present invention relates to a new method named the COBASORB test, which can be used for testing the cause of cobalamin malabsorption in humans. The COBASORB test contains three separate tests (first, second and third test) that can be performed separately, sequentially or in random order and number. The first test uses non-radioactive cobalamin for ingestion, the second test uses non-radioactive cobalamin and recombinant intrinsic factor for ingestion and the third test uses recombinant haptocorrin saturated with cobalamin for ingestion. All three tests involve analysis of changes in the concentration of cobalamin saturated transcobalamin (holo-TC) and cobalamin saturated haptocorrin (holo-HC) in the blood. Also disclosed are kits suitable for use in these methods.

All patent and non-patent references cited in the present application are hereby in-corporated by reference in their entirety.

### FIELD OF THE INVENTION

The present invention relates to the use of non-radioactive cobalamin for diagnosing the cause of vitamin B12 deficiency. The cobalamin can be used either in its free form or bound to proteins, e.g. intrinsic factor and haptocorrin. It is administered orally and then blood samples are analysed for changes in the concentration of co-balamin present in blood e.g. plasma cobalamin bound to transcobalamin and/or haptocorrin.

### BACKGROUND OF THE INVENTION

Vitamin B12 deficiency is a common condition occurring with a frequency of up to 10-15% in the elderly population. Absorption of cobalamin (vitamin B12) from the food is important for mammals as they need methyl- and 5′-deoxyadenosyl-cobalamin as a cofactor for two important enzymes, methionine synthase and methylmalonyl-CoA mutase. The transfer of cobalamin from the food to the blood involves intrinsic factor. Intrinsic factor is a cobalamin binding protein secreted in the stomach by the parietal cells of the gastric mucosa. Intrinsic factor binds to cobalamin in the intestine and the intrinsic factor-cobalamin complex is later absorbed by epithelial cells in the terminal ileum through binding to a receptor, cubilin. In the epithelial cell cobalamin is separated from intrinsic factor and transferred to the blood where it binds to transcobalamin and haptocorrin present in plasma. The transcobalamin-cobalamin complex and haptocorrin-cobalamin complex are referred to as holo-TC and holo-HC respectively. In many patients, vitamin B12 deficiency is caused by no or reduced secretion of intrinsic factor into the gastric juice. Ingestion of both intrinsic factor and cobalamin by these patients will cause a significant increase in the absorption of cobalamin.

The Schilling Test

The fact that absorption of cobalamin to the blood can be restored in patients with no intrinsic factor secretion simply by adding cobalamin together with intrinsic factor is used in a routine test, the Schilling test, employed in patient diagnosis of vitamin B12 deficiency (Ward, 2002). The aim is to determine whether the patient has a reduced secretion of intrinsic factor or an intestinal malabsorption of vitamin B12. The classical version of the Schilling test consists of two steps. In the first part, free radioactive cobalamin is ingested by the patient after having received an injection of a huge dose of unlabelled (non-radioactive) vitamin B12 in order to saturate the vitamin B12 binding proteins. This ensures that any absorbed labelled vitamin B12 is excreted in the urine. Urine is then collected over the next 24 hours and the amount of radioactive cobalamin present is determined. If very little radioactivity is present in the urine, this indicates a lack of cobalamin absorption, which may be caused by an intrinsic factor deficiency, such as a lack of intrinsic factor secretion, or by intestinal malabsorption. To distinguish between these two conditions the second part of the Schilling test is performed. In this part of the test the patient ingests radioactive cobalamin together with intrinsic factor. Again the urine is collected over the next 24 hours and the radioactivity determined. A significant increase of radioactivity in the urine supports the diagnosis that the patient suffers from a lack of intrinsic factor since the cobalamin absorption was restored by ingestion of cobalamin together with intrinsic factor. No radioactivity in the urine indicates that the patient has a defect further along the process of cobalamin absorption e.g. a malfunction of the intestine.

The Schilling test has been marketed in several modifications. One is to supply the labelled cobalamin built into food rather than in its free form. This has been done in order to test whether the patients' inability to absorb relates to a decreased capacity in liberating the vitamin B12 from food, such as it may be seen in patients suffering from pancreatic insufficiency.

Whatever the format of the Schilling test there are several severe problems and limitations attached to this method:
Most importantly is the use of labelled vitamin B12. Though the amount of radioactivity employed is limited (magnitude 0.5×10⁻⁶ ci) it is increasingly unacceptable both for the patient and for the clinical personnel handling the radioactive cobalamin and collecting the biological material needed for the test.
The collection of urine over a 24 hour period is problematic. It is time consuming and it is hampered by a relatively large uncertainty due to incomplete collection of the urine from the patient.
Other formats of the Schilling test involve stool collection (the Dicopac test) or whole body counting. These procedures are considered to be unpleasant and/or very time demanding.
The availability of intrinsic factor for use in the Schilling type tests is problematic, and currently there is no available source of human intrinsic factor.
The food cobalamin absorption test is hampered by lack of standardisation and because of this, the benefit of this test is limited.
The current Schilling type tests for vitamin B12 absorption does not allow an evaluation concerning the vitamin B12 status of the patient, that is in a patient able to absorb vitamin B12 the current tests are not able to clarify whether the patient is in need of additional vitamin B12.

Henze et al. (1988) have performed a study in which non-radioactive vitamin B12 was administered to patients and plasma vitamin B12 was determined. They found no significant difference between patients with a normal and patients with an abnormal Schilling test result. The authors concluded that the Schilling test cannot be carried out with non-radioactive vitamin B12.

Holo-TC Determinations

Holo-TC determination has been considered as a method for the identification of patients with cobalamin deficiency. However, as the physiological role of holo-TC is complex, it has been elusive what low holo-TC concentrations really indicate (Carmel, 2002)). Indeed, there has been no convincing evidence favouring either cobalamin deficiency or impaired absorption as the determinant of low holo-TC (Carmel (2002) Clinical chemistry 48, 407 and references therein).

### SUMMARY OF THE INVENTION

The inventors have established that serum levels of holo-TC reflect active vitamin B12 absorption. This has enabled the development of improved methods and kits for the determination of vitamin B12 absorption, using non-radioactive cobalamin.

The method of the present invention uses oral administration of non-radioactive cobalamin, human intrinsic factor and haptocorrin, and determination of changes in blood holo-TC and holo-HC concentrations. The present invention solves all of the above problems related to the Schilling test.

In the test of the present invention oral intake of non-radioactive cobalamin, either in its free form or complexed to human intrinsic factor or haptocorrin, is used. A blood sample is collected just before and at timed intervals after the intake of vitamin B12. Holo-TC in the samples is measured as an indicator of the uptake of vitamin B12 and/or holo-HC is measured as an indicator of the stores of vitamin B12. Recombinant proteins may be used in order to circumvent the risk of disease transmission and contamination with other vitamin B12 binding proteins, as may be a problem if native human proteins were to be used.

The present invention provides a number of advantages over the existing methods.
The present invention uses non-radioactive rather than radioactive vitamin B12.
The procedure involves just a simple blood test, as opposed to the collection of urine.
It requires little professional help. The patient ingests the cobalamin tablets with or without cobalamin binding protein and has a few blood samples taken.
It may use recombinant human intrinsic factor and haptocorrin produced in e.g. transgenic plants. Thus, transmission of human diseases and/or contamination with other cobalamin binding proteins is avoided.
The use of haptocorrin saturated with cobalamin, as a test dose of food cobalamin will allow standardisation of this part of the present invention.
The measurement of both holo-TC (reflects early changes in vitamin B12 absorption) and holo-HC (reflects stores of vitamin B12) allows a more refined diagnosis of patients suspected to suffer from vitamin B12 deficiency.

Since the Schilling test monitors the absorption of cobalamin by determination of the radioactivity in collected urine, the use of non-radioactive cobalamin in the test of the present invention demands another detection system. The plasma concentration of holo-TC and the saturation of TC increase after cobalamin absorption and the plasma concentration of holo-HC may increase after an additional period of time if the patient has sufficient stores of vitamin B12 in the body. Determination of plasma concentrations of holo-TC and/or holo-HC and/or determination of the saturation of TC and/or HC may therefore replace the measurement of radioactive cobalamin in the urine.

The test as described herein involves taking blood samples before and after ingestion of preferably several times the recommended daily dose of cobalamin followed by analysis of the blood samples for changes in holo-TC and holo-HC concentrations. After preferably a few days the patient may ingest similar amounts of cobalamin but now together with intrinsic factor. New blood samples are taken and analysed for changes in holo-TC and holo-HC concentrations. Combination of the results from the two sets of blood analyses makes it possible to diagnose whether a lack of intrinsic factor or intestinal malabsorption is the cause of vitamin B12 deficiency. A low holo-HC concentration in the blood indicates that the patient has transported all of the vitamin B12 into the cells of the body and that no or little has returned to the blood. Oral administration of haptocorrin-bound cobalamin makes it possible to investigate the ability of the patient to transfer cobalamin from the food to intrinsic factor.

Therefore the present invention has a number of advantages compared to the traditional Schilling test:
It does not involve orally administration of radioactive cobalamin.
It eliminates the risk( of loss of radioactive urine or stools and contamination of surroundings.
It uses blood samples instead of collected urine. Collection of blood samples is simple whereas the collection of urine can be very problematic for many patients resulting in loss of urine and incorrect calculation of cobalamin absorption as in the Schilling test.
It may determine the holo-TC concentration and/or the TC saturation before and after ingestion of cobalamin, or cobalamin plus intrinsic factor, or cobalamin plus haptocorrin. Therefore, the test allows a standardised assay for testing both the ability of intrinsic factor to restore the absorption of vitamin B12 and for the ability of the patient to handle vitamin B12 bound to a protein believed to be representative for the protein binding of vitamin B12 in food.
It can include determination of holo-HC concentrations and/or HC saturation in the blood samples. These results give information about the cobalamin status over a long period in the patient. The Schilling test gives no such information.
The test may use large doses of cobalamin in its free form or bound to intrinsic factor compared to the doses used in the Schilling test. This may give information about the intestinal absorption capacity.

### DESCRIPTION OF DRAWINGS

FIG. 1: The changes in serum vitamin B12 (▪), total TC (▴), holo-TC (●) and TC saturation (♦) in 31 healthy subjects after ingestion of 3 times 9 μg of vitamin B12.

The percent increase from baseline (day 0) is indicated. Mean and standard error of the mean are shown. There were highly significant changes in all parameters from baseline values (day 0) to day 1 (p<0.0002 for all parameters) or 2 (p<0.0005 for all parameters, except TC (p=0.002) after intake of vitamin B12. On day 6 only the change of vitamin B12 was significantly different from the baseline value (p=0.0024).

FIG. 2: Individual plots of percentage increase of TC saturation (A), and vitamin B12 (B) from baseline at timed intervals after oral intake of vitamin B12 in 31 healthy subjects.

The horizontal lines represent a minimum increase (21%) for TC saturation among responders on day 1 (n=30).

FIG. 3: The absolute changes in serum vitamin B12, total TC, holo-TC and TC saturation in 31 healthy subjects (∘) and 7 patients (▴=diagnosed as Crohn's disease, ●=diagnosis is unclear) after ingestion of 3 times 9 μg of vitamin B12. The results of the Schilling test I (urinary excretion of radioactive vitamin B12 over 24 hours) for seven patients are presented on the graph. Urinary excretion of 10-40% of administered dose Is considered normal.

Differences observed after vitamin B12 intake (day 1-day 0) for each parameter were plotted against the initial value (day 0) for the corresponding parameter. Thin vertical lines represent the lower reference value for each parameter. Thin horizontal lines represent the minimum increase for holo-TC (15 pmol/L), and TC saturation (0.02) in control patients after omitting outliers (n=2). The thin horizontal line for total TC represents 0. The thin horizontal line for vitamin B12 represents the minimum increase observed for holo-TC (15 pmol/L).

### DETAILED DESCRIPTION OF THE INVENTION

### METHODS OF THE INVENTION

In a first aspect, the present invention provides a method to determine the cause of vitamin B12 deficiency in a patient which comprises comparing the concentration of holo-TC and/or holo-HC in the blood or serum following ingestion of non-radioactive cobalamin or analogues thereof, with the concentration in a sample taken prior to said ingestion.

Similarly, the invention relates to a method for diagnosing a vitamin B12 deficiency in an individual comprising the steps of:
i) obtaining a blood sample from an individual,
ii) having said individual ingest a dose of non-radioactive cobalamin or an analogue thereof, together with a binding protein or without a binding protein,
iii) obtaining, after a time period sufficient to allow uptake, if any, of the cobalamin or analogue thereof in the blood stream, a second blood sample from said individual,
iv) determining in said two samples one or more selected from the group consisting of: the concentration of holo-TC, the concentration of holo-HC, the saturation of TC, and the saturation of HC, and
v) determining, on the basis of comparison of said concentration and/or saturation in said two samples, whether said cobalamin or analogue thereof has been absorbed in the blood stream.

Furthermore, the invention relates to a method for determining absorption of vitamin B12 in an individual comprising the steps of:
i) providing two blood samples from said individual, wherein the first sample was taken before ingestion by said individual of non-radioactive cobalamin or an analogue thereof, together with binding protein or without a binding protein, and
   the second sample was taken after said ingestion,
ii) determining in said samples one or more selected from the group consisting of: the concentration of holo-TC, the concentration of holo-HC, the saturation of TC, and the saturation of HC, and
iii) determining, on the basis of comparison of said concentration and/or saturation in said two samples, whether said cobalamin or analogue thereof has been absorbed in the blood stream.

Accordingly, in some embodiments, the determination consists of determination of the holo-TC concentration and/or the TC saturation. In other embodiments, it consists of determination of the holo-HC concentration and/or the HC saturation. The determination may in yet other embodiments consist of determination of all four parameters, i.e. the concentration of holo-TC, the concentration of holo-HC, the saturation of TC and the saturation of HC.

In a preferred embodiment, active (i.e. intrinsic-factor-mediated) absorption is determined.

The time passing between the ingestion of non-radioactive cobalamin or analogues thereof and the taking of the subsequent blood sample must be long enough to allow uptake (if any) of the non-radioactve cobalamin or analogues thereof in the blood stream.

Preferably, the first blood sample, for establishing the holo-TC and/or holo-HC concentration and/or the saturation of TC and/or HC before absorption of the ingested dose of cobalamin is taken before the ingestion. However, the expression “taken before ingestion” is also intended to encompass the situation wherein the first blood sample is taken simultaneously with the ingestion, or immediately after the ingestion before absorption can have taken place.

The method can be modified by the ingestion of intrinsic factor or haptocorrin with the cobalamin. Two or more versions of the tests can be carried out in a patient, sequentially, in any order. For example all three versions of the test can be carried out by the patient ingesting cobalamin alone in the first test, cobalamin and intrinsic factor in the second test, and cobalamin and haptocorrin in the third test.

Cobalamin (vitamin B12) is a molecule that consists of a corrin ring with four pyrrole units, which surround and bind to the essential and central cobalt atom. Below the corrin plane is a nucleotide derivative with a dimethylbenzimidazole base, which also is linked to the cobalt atom. Finally the cobalt atom binds to a sixth molecule (e.g.: —CH3, —OH, —H2O, 5′-deoxyadenosyl, —CN) located above the corrin plane. In the present application we use the terms “cobalamin” and “vitamin B12” to indicate any form of the vitamin that in the human being can be converted to the active forms of the vitamin. Cobalamin cannot be synthesised by animals or plants and is only produced by some microorganisms, in particular, anaerobic bacteria. The term “cobalamin” as used herein includes cobalamin, cyano-cobalamin, methyl-cobalamin, hydroxy-cobalamin or analogues thereof with a capacity for binding to intrinsic factor, transcobalamin, and/or haptocorrin.

The cobalamin used for oral administration is a non-radioactive form. The purpose of the administration of cobalamin may be therapeutic or non-therapeutic. One, two, three or more doses can be taken at regular intervals, for example every six hours. Repeated ingestion of cobalamin may increase the concentration of holo-TC and possibly also holo-HC in the blood if absorption of cobalamin occurs. Administration of several times the recommended daily dose of cobalamin will result in a significant increase of the holo-TC concentration in the blood, if the absorption system works well. Use of small doses of cobalamin (less than 0.5 nano-mole), as in the Schilling test will not give a significant increase in holo-TC in the blood. Preferably, the dose is chosen such that passive absorption (i.e. absorption not mediated by intrinsic factor) is minimised. Thus, preferably, the total ingested dose of cobalamin is between 0.5 and 500 nanomole, more preferably between 1 and 250 nanomole, even more preferably between 2 and 100 nanomole, most preferably between 5 and 50 nanomole. In a particularly preferred embodiment, three doses of cobalamin are ingested, each being between 5 and 15 nanomoles.

Blood samples taken some hours e.g. the next morning after ingestion of cobalamin favours a maximal change in holo-TC concentration in the blood if cobalamin can be absorbed from the intestine and transferred to TC in the blood. In one preferred embodiment the concentration of holo-TC and/or holo-HC and/or total-TC and/or total-HC in the blood is measured less than 48 hours, more preferably 8-16 hours, after the last ingestion of cobalamin. If two or more versions of the test are to be carried out in the same patient (e.g. following ingestion of cobalamin alone, and/or ingestion of cobalamin with haptocorrin, and/or ingestion of cobalamin with intrinsic factor) in the second test, the initial concentration of holo-TC and/or holo-HC and/or total-TC and/or total-HC in the blood more than 48 hours, preferably 5-10 days after the last administration of cobalamin is measured.

The cobalamin binding proteins are proteins capable of binding cobalamin or analogues thereof. The cobalamin binding proteins used in this invention are transco-balamin, intrinsic factor and haptocorrin or functional equivalents of any one of these proteins. Functional equivalents herein means having retained the ability to bind cobalamin. Functional equivalents can e.g. be functionally equivalent fragments or functionally equivalent variants of the cobalamin binding proteins. Preferred fragments of a cobalamin binding protein comprise at least 50%, such as at least 75%, such as at least 90% of the total length of the corresponding protein. Preferred variants have at least 50%, such as at least 75%, such as at least 90% sequence identity to the corresponding protein.

The percent identity of two amino acid sequences is determined by aligning the sequences for optimal comparison purposes (e.g., gaps can be introduced in both sequences for best alignment) and comparing the amino acid residues at corresponding positions. The “best alignment” is an alignment of two sequences, which results in the highest percent identity. The percent identity is determined by the number of identical amino acid residues in the sequences being compared (i.e., % identity=number of identical positions/total number of positions×100).

The cobalamin binding proteins used for ingestion and analysis of plasma holo-TC and plasma holo-HC concentrations may be native e.g. from human, pig or recombinant cobalamin binding proteins produced in e.g. yeast, plants, plant cells, insect cells, mammalian cells. The cobalamin binding proteins are preferably recombinant human proteins produced by yeast or transgenic plants since this will eliminate the risk of transferring mammalian pathogens from sources of intrinsic factor and haptocorrin that contain other mammalian material.

The cobalamin, intrinsic factor and haptocorrin are all adapted for oral administration. They may be presented as discrete units such as capsules or tablets; powders or granules; solutions or suspensions in aqueous or non-aqueous liquids; edible foams or whips; or oil-in-water liquid emulsions or water-in-oil liquid emulsions.

The methods described herein may be carried out on samples from all types of individuals including healthy individuals, individuals suspected of suffering from a vitamin B12-related deficiency but not having been diagnosed yet, or patients known to suffer from a vitamin B12-related deficiency. In one embodiment, the individual is not suffering from AIDS.

The holo-TC and holo-HC concentrations in the blood can be determined by several different methods. A few of such methods are described below, but any suitable method can be used. Suitable methods are, for example, the ones described in US patent applications US20010051346 and US20030148541.

The concentration of total transcobalamin (both apo- and holo-TC) and total haptocorrin in the blood sample can be determined by e.g. ELISA using antibodies against transcobalamin and haptocorrin respectively. The fraction of transcobalamin and haptocorrin in the apo-form (not saturated with cobalamin) can be separated by affinity to beads or a solid material coated with cobalamin. Then the amount of bound TC and HC can be determined by ELISA with antibodies against transcobalamin and haptocorrin. The concentration of holo-TC and holo-HC can be calculated by subtraction of the concentration of apo-form from the concentration of both apo- and holo-form. TC saturation and HC saturation can be calculated as holo-TC/total-TC and holo-HC/total-HC, respectively.

Alternatively the holo-form concentration of transcobalamin or haptocorrin in the blood samples can be determined by, for example, ELISA utilising monoclonal antibodies that only recognise the holo-form but not the apo-form or cobalamin alone.

Alternatively, the holo-form concentration of transcobalamin and haptocorrin can be determined using a method described by Nexø et al. (2002). This method involves removal of the apo-TC and apo-HC from the sample by cobalamin coated magnetic beads that will bind the apo-form of cobalamin binding proteins. The beads are removed so that the supernatant now contains holo-TC and holo-HC plus other proteins not able to bind cobalamin but no apo-form of TC and HC. The concentration of holo-TC and holo-HC is determined by using ELISA with antibodies against TC and HC respectively as described for TC by Nexøet al. (2000).

The concentration of holo-TC in the blood can also be determined by using a radio-binding assay, such as the holo-TC RIA from Axis Shield (Norway) and described by Uleland et al. (2002).

An increase of the holo-TC concentration and/or the TC saturation in the blood following ingestion of cobalamin reflects that the tested person secretes intrinsic factor, which is able to bind the ingested cobalamin. It also indicates that the cobalamin-intrinsic factor complex was able to bind to the intestinal receptor and finally transfer of the cobalamin to transcobalamin in the blood occurred. Alternatively, if there is a small or no increase in holo-TC concentration and/or TC saturation, this indicates that the person has little or no secretion of intrinsic factor for efficient transport of cobalamin to the intestinal receptor or the capacity of intestinal absorption is limited e.g. because of problems with the intestinal receptors. The use of several times the recommended daily dose of cobalamin will saturate the absorption system and therefore give an indication of the capacity for absorption.

The absorbed cobalamin will appear first in complex with TC in the blood and later it will be transferred to HC. Therefore the holo-HC concentration will increase later than the holo-TC concentration in the blood. If the tested person has suffered from vitamin B12 deficiency for a long period the stores of cobalamin in the organism will be low or empty. Absorption of a few nano-moles of cobalamin will cause a temporary increase in holo-TC before the cobalamin is transferred to the tissue cells. In this situation the holo-HC concentration in the blood will not increase significantly.

The intrinsic factor or haptocorrin and cobalamin can be taken together, separately, or sequentially. To facilitate comparison of the results from the first version of the method (e.g. ingestion of only cobalamin) with the results of the second and/or third version of the method (ingestion of both cobalamin and intrinsic factor and/or haptocorrin) it is preferred that the dose of cobalamin used is equal in all of the tests.

When orally administered cobalamin is not absorbed by the intestine, the cause of vitamin B12 deficiency may be lack of intrinsic factor secretion or another type of intrinsic factor deficiency. Therefore both cobalamin and intrinsic factor are orally administered to the patient. If an increase in blood holo-TC and holo-HC follows after ingestion of both cobalamin and intrinsic factor the patient suffers from insufficient intrinsic factor secretion or another type of intrinsic factor deficiency. A small or a large increase in holo-TC reflects a small and a large capacity respectively from the intestinal cobalamin-intrinsic factor absorption. An increase in holo-TC but no increase in holo-HC reflects that the patient has suffered from vitamin B12 deficiency for a long period of time resulting in small or no body stores of holo-HC.

The method can be adapted to determine whether the vitamin B12 deficiency is caused by lack of transfer of cobalamin from haptocorrin (as surrogate for food with protein bound cobalamin) to intrinsic factor in the intestine.

When the patient is able to absorb cobalamin after ingestion of cobalamin but still suffers from vitamin B12 deficiency, the cause of deficiency is not lack of intrinsic factor or intestinal receptors but possibly lack of ability to transfer cobalamin from the cobalamin binding proteins in the food to intrinsic factor. No increase in holo-TC concentration in the blood following ingestion of several times the recommended daily dose of cobalamin bound to haptocorrin (or another binding protein that can serve as surrogate for cobalamin binding proteins in food) indicates that the person is unable to liberate vitamin B12 from the food such as in patients suffering from pancreatic malfunction.

### KITS OF THE INVENTION

In a further aspect the present invention provides kits for use in the diagnosis of vitamin B12 deficiency. Thus, the invention relates to a kit-of-parts suitable for use in the diagnosis of a vitamin B12-related deficiency, comprising
i) materials suitable for determining the holo-TC and/or holo-HC concentration in a blood sample, and
ii) instructions to the user comprising a description of the possible use of the kit in carrying out any of the methods defined herein.

Furthermore, the invention relates to a kit-of-parts suitable for use in the diagnosis of vitamin B12 deficiency comprising non-radioactive cobalamin and antibodies to transcobalamin and/or antibodies to haptocorrin.

These kits may comprise one or more containers containing e.g. any one or more of cobalamin, intrinsic factor, haptocorrin, antibodies to transcobalamin, antibodies to haptocorrin, cobalamin bound to beads or a solid support, buffers and columns. The antibodies may also be monoclonal antibodies that only recognise holo-TC, and not the apo-TC or cobalamin. A labelled form of a cobalamin binding protein may also be present. The components can be provided as individual components or a ready prepared mixture. The reagents may be provided in a freeze-dried or lyophilised form or as a ready made solution. Such kits may also include other containers or devices for utilising the kit.

### EXAMPLES

A study was performed to evaluate whether changes in holo-TC and/or TC saturation reflect vitamin B12 absorption.

Materials and Methods

The subjects participating in the study were 31 healthy subjects recruited in October 2002. None of them suffered from known disorders related to vitamin B12 deficiency. Persons with chronic systemic disease, persons taking any kind of medical treatment, including vitamin tablets within the past week and persons not being able to give written informed consent were excluded. The age of the healthy subjects ranged from to 25 to 57 (mean 40) years. There were 9 men and 22 women. We further included seven patients (age 22-39 years, five men and two women) who had been referred to the out-patient clinic of internal medicine department during 2003 because vitamin B12 malabsorption was suspected. Three of the seven patients have previously been diagnosed as Crohn's disease. The diagnosis of the remaining four patients was not clear. Written informed consent was obtained from all subjects, and the Research Ethics Committee of Aarhus County approved the study protocol (2002.0224).

### Study Protocol

The absorption of vitamin B12 was evaluated from analysis of serum vitamin B12, total TC, and holo-TC on samples obtained before and after oral administration of vitamin B12.

In healthy subjects samples were taken at 8:00 a.m. on the day before vitamin B12 intake (−1) and on day 0, 1, 2, and 6. After the blood sample was removed on day 0, an oral dose of 9 μg vitamin B12 (Natur Drogeriet A/S, Hoerning, Denmark) was given three times, with 6 h between the doses (8 a.m., 2 p.m., and 8 p.m. (time points were allowed to deviate ±45 min). One healthy subject was unavailable for delivering a blood sample on day 6. The absorption of vitamin B12 in seven patients was evaluated by the Schilling test I and by the design mentioned above except that the blood samples were obtained only on day 0 and day 1. The Schilling test I was performed after our alternative approach.

The vitamin B12 tablets were given with either water or orange juice. The subjects were allowed to have a light breakfast 30 to 60 min before blood sampling, not including any diary products, but were otherwise allowed to eat their normal diet. The blood samples were centrifuged within 60 minutes and were stored at −80° C. until further processed.

The Schilling Test I

The Schilling test I was performed as described previously (Chanarin, 1979). Briefly, a fasting patient is given a 1 μg oral dose of vitamin B12, which is tagged with radioactive cobalt (Co-57). Two hours after the oral dose the patient is then injected intramuscularly with 1000 μg of non-labeled vitamin B12. A 24 hour urine collection is initiated. The percentage of the administered dose excreted in the urine over 24 hours is then determined. Urinary excretion of 10-40% of the administered dose is considered normal.

Biochemical Analysis

Serum vitamin B12 was determined by a commercial method (Bayer corporation, NY) on a Centaur equipment (analytical imprecision<10%). Serum total TC and holo-TC were measured by ELISA as recently described (Nexø et al., 2000; 2002), but modified to allow the use of an automated ELISA analyser (BEP-2000, Dade Behring, Germany). The following modification was performed; all incubations were performed at 37° C. The analytical imprecision was 7% for total TC (mean=934 pmol/L, n=91) and 8% for holo-TC (mean=38 pmol/L, n=41). The controls were run over 12 months for total TC and 6 months for holo-TC. The reference interval was established from analysing 161 samples obtained from healthy blood donors (age interval; 21-65). The reference interval was 700-1400 pmol/L for total TC, ≧50 pmol/L for holo-TC and ≧0.05 for TC saturation. Haematological parameters were analysed on the Coulter Counter (Beckman Coulter CA). Plasma creatinine was measured using the Jaffe method and a Roche Cobas integra 700 autoanalyzer (analytical imprecision <3%).

Statistical Analysis

The intra-individual variation was calculated using estimation of variance by ANOVA from the measurements of the analytes from the two samples obtained before the treatment (day-1 and day 0).

Alterations (increase or decrease) in parameters as a function of time were analysed by comparing the changes obtained on the same individuals relative to baseline (day 0) with the theoretical median “0” assigned for day 0. Since the data did not present normal distribution, non-parametric testing (Wilcoxon matched pair test) was employed. P-values <5% were regarded as statistically significant. Data were analysed using SPSS10.0 (SPSS Inc.) and the GraphPad (Prism2) software.

Results

All 31 healthy subjects had normal erythrocyte count, haemoglobin, mean cell volume and creatinine levels as summarised in Table 1.

TABLE 1
Median and range, and intra-individual variation for vitamin B12,
serum holo-TO, total-TC and TC saturation in 31 healthy subjects ^{a}.

| | | |
|---|---|---|
| Range | Reference | Varia- |
| (median) | Interval ^{b} | tion ^{c} |

| | Baseline | Male | Female | (%) |
|---|---|---|---|---|
| Age, years | 25-57 (40) | — | — | |
| Blood hemoglobin, mmol/L | 7.8-10.2 | 8.4-10.8 | 7.4-9.6 | |
| | (8.5) | | | |
| Mean Cell volume, fL | 79-98 (89) | 85-100 | 85-100 | |
| Erythrocyte count, 10¹²/L | 3.9-5.6 (4.4) | 4.1-6.1 | 3.7-5.5 | |
| Plasma creatinine, μmol/L | 61-106 (78) | 62-133 | 44-115 | |

| | | | |
|---|---|---|---|
| Holo-TC, pmol/L | 36-281 (73) | ≧50 | 11 |
| Total-TC, pmol/L | 747-1471 | 700-1400 | 8 |
| | (947) | | |
| TC saturation, fraction ^{d} | 0.02-0.22 | ≧0.05 | 13 |
| | (0.08) | | |
| Vitamin B12, pmol/L | 163-661(250) | 200-650 | 6 |

^{a} Laboratory parameters were determined from the blood samples obtained day −1.
^{b} Intervals of references for holo-TC, total-TC and TC saturation was based on analyses of 161 samples obtained from healthy blood donors.
^{c} Intra-individual variation was calculated based on values obtained on day −1 and day 0 from the 31 healthy subjects before receiving B12.
^{d} Calculated as holo-TC/total-TC.

The intra-individual variation was below 13% for all parameters (Table 1), as calculated from data obtained on the samples collected prior to the intake of vitamin B12 (day-1 and day 0).

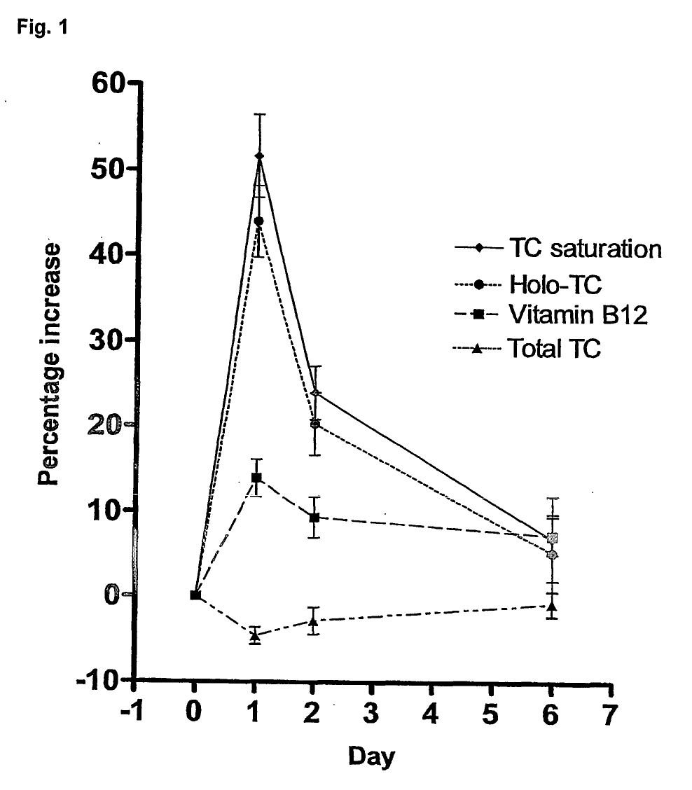

After oral intake of three times 9 μg of vitamin B12, all parameters studied changed as indicated in FIG. 1. The changes relative to baseline (day 0) were highly significant on day 1 (p<0.0002 for all parameters) and day 2 (p<0.0005 for holo TC, TC saturation and vitamin B12, p-0.02 for total TC). The maximal percentage and absolute increase (median and (range)) in holo-TC was 39 (0-+108) %, 34 (0-149) pmol/L, and in TC saturation 52 (−2-+128) %, 0.04 (0-0.23) as a fraction, respectively (n=31). A maximal increase of 15% or more for holo-TC and TC saturation was observed at day 1 for 29 subjects and at day 2 for one subject. Only one healthy subject did not increase in holo-TC and TC saturation.

The percentage and absolute increase in serum vitamin B12 was less dramatic (14 (−8-+51)) %, 36 (−27-290) pmol/L. Four healthy subjects did not increase and 14 healthy subjects increased less than 15%.

Small though significant changes were observed for total TC. The maximal percentage and absolute decrease were 5 (−16-+9)) % and 46 (−180-+77) pmol/L. Twenty-three of the 31 healthy subjects showed a decrease in total TC concentration at day 1.

After 1 day, the highest levels (median (range)) were obtained for holo-TC (118 (56-344)) pmol/L, TC saturation (0.13(0.06-0.43)) and serum vitamin B12 (279 (176-856)) pmol/L (Table 2). After 6 days the levels for holo-TC, total TC and TC saturation did not differ significantly from baseline, while the level of serum vitamin B12 remained significantly higher than baseline (p=0.0086).

TABLE 2
Absolute values (median and range) obtained for TC saturation, holo
TC and vitamin B12 before (day 0) and at timed intervals after oral
intake of vitamin B12 in 31 healthy subjects.

| | Median | | | |
|---|---|---|---|---|
| | (Range) | | | |
| | DAY 0 | DAY 1 | DAY 2 | DAY 6 |
| Holo-TC, | 72 | 118 | 87 | 80 |
| (pmol/L) | (39-298) | (56-344) | (41-319) | (37-302) |
| Total-TC, | 905 | 855 | 843 | 885 |
| (pmol/L) | (734-1599) | (710-1526) | (687-1390) | (717-1024 |
| TC saturation | 0.08 | 0.13 | 0.11 | 0.09 |
| (fraction) | (0.03-0.26) | (0.06-0.43) | (0.04-0.32) | (0.04-0.27) |
| VitaminB12, | 234 | 279 | 253 | 260 |
| (pmol/L) | (154-566) | (176-856) | (172-830) | (174-627) |

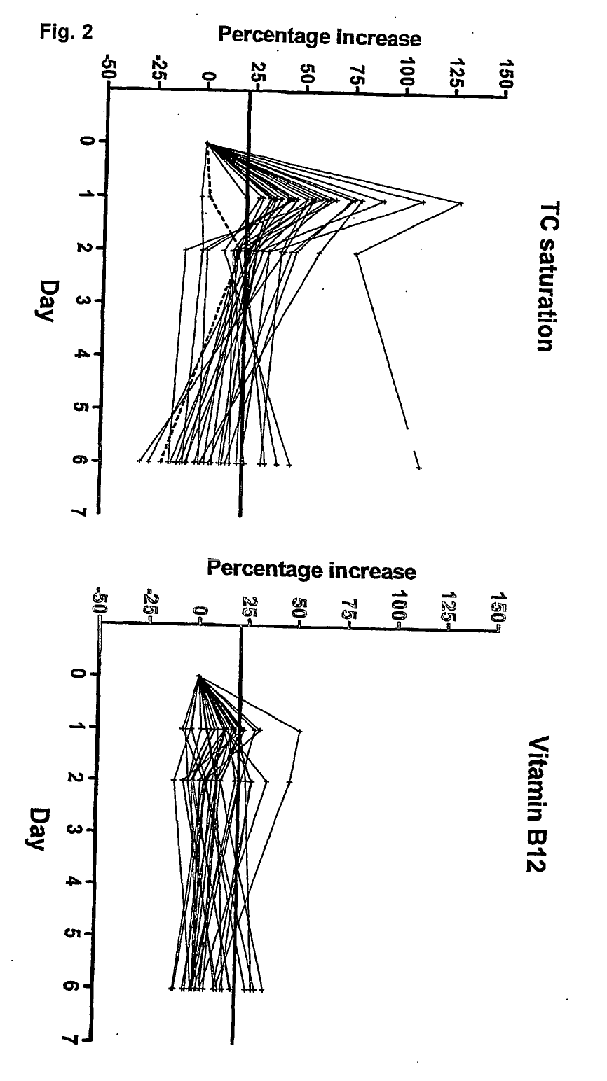

The calculated TC saturation becomes a slightly better marker for vitamin B12 absorption because of the observed decrease in total TC together with the increased holo-TC concentration after vitamin B12 intake. All, but one healthy subject showed an increase of 21% or more in TC saturation. However, only seven healthy subjects showed such an increase or more in serum vitamin B12 concentration (FIG. 2).

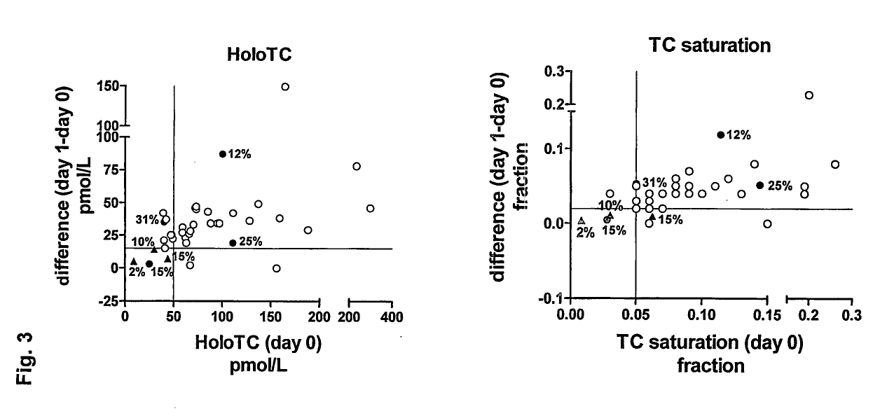

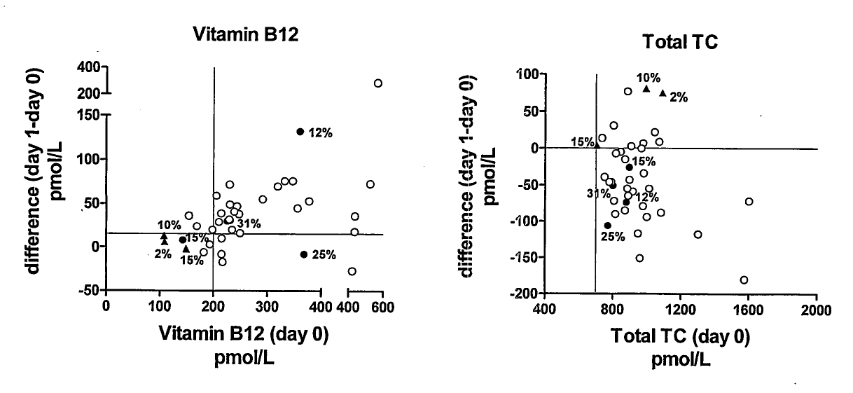

Four of the seven patients suspected to have decreased vitamin B12 absorption presented serum holo-TC and vitamin B12 values lower than the reference interval (FIG. 3), though their haematological parameters were normal (data not shown). Three of these four patients were previously diagnosed as having Crohn's disease. After vitamin B12 intake, these three patients with Crohn's disease presented negligible increase in holo-TC (3, 7, 14 pmol/L) and TC saturation (0.004, 0.01, 0.01), (FIG. 3).

For all seven patients, except one, the quantitative results of the Schilling test I (percentage of vitamin B12 excreted in the urine) were roughly comparable with the change of serum holo-TC after vitamin B12 intake (FIG. 3). One of three patients with Crohn's disease had an abnormal Schilling test 1 (2%). The other two had Schilling test I in the normal range (10-40%), but their values were in the lower part of the reference interval (10%, 15% respectively).

### DISCUSSION

This study documents that serum levels of holo-TC and TC saturation reflect the active vitamin B12 absorption. One day after an oral dose of three times 9 μg vitamin B12 the level of holo TC and TC saturation increased with a median value of around 50%, whereas the increase was only 14% for serum vitamin B12 in healthy subjects. These findings strongly suggest that measurement of holo-TC and/or TC saturation after an oral dose of vitamin B12 holds more information than measurement of serum vitamin B12 when it comes to evaluate active absorption of vitamin B12.

So far little attention has been paid to the dose of vitamin B12 administered to the patient in order to study the active uptake of vitamin B12 by use of blood tests. Most studies performed so far have used a relatively larger single oral dose of vitamin B12 (1000 μg) (Henze et al. 1988; Moridani et al. 2002). The crucial point here is that with this large dose of vitamin B12, the non-IF mediated absorption of 1% alone will raise the plasma concentration thereby falsifying the measurement. This increase does not reflect active IF mediated absorption and thus has limited diagnostic impact as regards the active vitamin B12 absorption.

In our study we designed the intake of vitamin B12 to meet two criteria. Firstly, we wanted to minimise passive absorption, accounting for approximately 1% of the dose supplied (Chanarin, 1979). Secondly, we wanted to accumulate as much actively absorbed vitamin B12 as possible in order to get an optimal signal. To meet these two demands, we chose to use a high physiological dose (9 μg) and to administer this dose three times with 6 hours interval. It is well known that after the ingestion of a dose of vitamin B12 there will be a refractory phase about 3 hours with less absorption of vitamin B12 as far as further vitamin B12 uptake is concerned (Chanarin, 1979). A further dose of vitamin B12 is absorbed normally when given about 4-6 h after the initial dose (Chanarin, 1979). It has previously been shown that the highest amount of IF bound vitamin B12 was obtained if a dose of 10 μg vitamin B12 was employed.

### REFERENCES

Chanarin I. The megaloblastic Anaemias. Oxford: Blackwell Scientific Publications, 1979.

Nexo E, Christensen A L, Petersen T E et al. Measurement of transcobalamin by ELISA. Clin Chem 2000; 46:1643-9.

Nexo E, Christensen A, Hvas A M et al. Quantification of holo-transcobalamin, a marker of vitamin B12 deficiency. Clin Chem 2002; 48:561-2.

Henze E, Manner S, Clausen M et al. The Schilling test cannot be replaced by an absorption test with unlabeled vitamin B12. Klin Wochenschr 1988; 66:332-6.

Moridani, M. Y., Hoffman, B., Pritzker, K, and Bromberg, I. Vitamin B12 absorption test: (Abstract). Clin Chem 2002; 48:A146.

Uleland M, Eilertsen I, Quadros E V et al. Direct assay for cobalamin bound to transcobalamin (holo-transcobalamin) in serum. Clin Chem 2002; 48:526-32.

Nexo E, Hvas A M, Bleie O et al. Holo-transcobalamin is an early marker of changes in cobalamin homeostasis. A randomized placebo-controlled study. Clin Chem 2002; 48:1768-71.

Carmel R. (2002) Measuring and interpreting holo-transcobalamin (holo-transcobalamin II) Clin. Chem; 48: 407409.

1-41. (canceled)

42. A method for determining absorption of vitamin B12 in an individual comprising the steps of:
i) providing two blood samples from said individual, wherein the first sample was taken before ingestion by said individual of non-radioactive cobalamin or an analogue thereof, together with binding protein or without a binding protein, and
the second sample was taken after said ingestion,
ii) determining in said samples one or more selected from the group consisting of: the concentration of holo-TC, the concentration of holo-HC, the saturation of TC and the saturation of HC, and
iii) determining, on the basis of comparison of said concentration and/or saturation in said two samples, whether said cobalamin or analogue thereof has been absorbed in the blood stream.

43. The method according to claim 42, wherein said non-radioactive cobalamin or analogue thereof was ingested without a binding protein.

44. The method according to claim 42, wherein said non-radioactive cobalamin or analogue thereof was ingested together with intrinsic factor or an analogue, fragment or variant thereof.

45. The method according to claim 44, wherein said intrinsic factor or analogue, fragment or variant thereof is of recombinant origin.

46. The method according to claim 44, wherein said intrinsic factor or analogue, fragment or variant thereof is derived from a recombinant plant.

47. The method according to claim 42, wherein said non-radioactive cobalamin or analogue thereof was ingested together with
haptocorrin or an analogue, fragment or variant thereof or
another binding protein that can serve as surrogate for cobalamin binding proteins in food.

48. The method according to claim 47, wherein said haptocorrin or analogue, fragment or variant thereof or said other binding protein is of recombinant origin.

49. The method according to claim 47, wherein said haptocorrin or analogue, fragment or variant thereof or said other binding protein is derived from a recombinant plant.

50. The method according to claim 43, further comprising the step of ingesting: intrinsic factor or an analogue, fragment or variant thereof.

51. The method according to claim 42, wherein two or more doses of cobalamin (together with a binding protein or without a binding protein) are ingested.

52. The method according to claim 51, wherein the cobalamin was ingested three times at six hours intervals.

53. The method according to claim 42, wherein the total ingested dose of cobalamin was between 0.5 and 500 nanomole.

54. The method according to claim 52, wherein each of the three ingested doses was between 5 and 15 nanomole.

55. The method according to claim 42, wherein the determination of one or more selected from the group consisting of:
the concentration of holo-TC, the concentration of holo-HC, the saturation of TC, and the saturation of HC,
is performed less than 48 hours after the last ingestion of cobalamin.

56. The method according to claim 55, wherein the determination of one or more selected from the group consisting of:
the concentration of holo-TC, the concentration of holo-HC, the saturation of TC, and the saturation of HC,
is performed 8-16 hours after the last ingestion of cobalamin.

57. The method according to claim 50, wherein the initial determination of one or more selected from the group consisting of:
the concentration of holo-TC, the concentration of holo-HC, the saturation of TC, and the saturation of HC,
for the subsequent method is performed more than 48 hours after the last ingestion of cobalamin in the previous method.

58. The method according to claim 57, wherein the initial determination of one or more selected from the group consisting of:
the concentration of holo-TC, the concentration of holo-HC, the saturation of TC, and the saturation of HC,
for the subsequent method is performed 5-10 days after the last ingestion of cobalamin in the previous method.

59. The method according to claim 50, wherein the cobalamin, together with or without the binding protein, was ingested in the same amount, in the same number of doses, with the same number of hours between doses in each method.

60. The method according to claim 50, wherein the determination of one or more selected from the group consisting of:
the concentration of holo-TC, the concentration of holo-HC, the saturation of TC, and the saturation of HC,
in the blood is performed at the same time after the ingestion of cobalamin, together with or without binding protein, in each method.

61. The method according to claim 42, wherein the concentration of holo-TC and/or holo-HC is measured by immunosorbent assay.

62. The method according to claim 61, wherein the immunosorbent assay is an ELISA or RIA.

63. The method according to claim 61, wherein the holo-TC and/or holo-HC concentration is determined by subtracting the concentration of apo-TC and/or apo-HC from the total concentration of TC and/or HC.

64. The method according to claim 61, wherein the apo-TC and/or apo-HC are removed by passing the sample over cobalamin complex to a solid phase support.

65. The method according to claim 63, wherein the concentration of apo-TC and/or apo-HC is determined by using monoclonal antibodies specific for apo-TC or apo-HC.

66. The method according to claim 61, wherein the concentration of holo-TC and/or holo-HC is determined by using monoclonal antibodies specific for holo-TC or holo-HC.

67. The method according to claim 42 wherein the holo-TC or holo HC concentration is determined by measurement of the vitamin B12 attached to holo-TC or holo-HC.

68. The method according to claim 67, wherein the determination comprises the following steps:
separation of both apo- and holo-form of TC or HC from the sample by binding to antibodies against TC or HC;
release of the cobalamin from the holo-TC or holo-HC fraction and removal or destruction of the TC or HC;
determining the amount of cobalamin released by a competitive binding assay.

69. The method according to claim 42, wherein the final determination step comprises determining whether any one or more selected from the group consisting of:
the holo-TC concentration, the holo-HC concentration, the TC saturation and the HC saturation,
in the second sample is higher than in the first sample.

70. A method for the evaluation of a possible vitamin B 12-related deficiency in an individual comprising the steps of the method of claim 42, and further comprising the step of evaluating, on the basis of comparison of said concentration and/or saturation in said samples, whether said individual suffers from a vitamin B12-related deficiency and/or what the cause of said deficiency is.

71. A method for determining the cause of a vitamin B12-related deficiency in an individual comprising the steps of the method of claim 42, and further comprising the step of evaluating, on the basis of comparison of said concentration and/or saturation in said samples, what the cause of said deficiency is.

72. A method for evaluating whether a vitamin B12-related deficiency is due to an intrinsic factor deficiency, such as lack of intrinsic factor secretion, or to a malabsorption of intrinsic-factor-bound cobalamin in the intestine, said method comprising the carrying out the method of claim 44, and further comprising the step of evaluating, on the basis of comparison of said concentration and/or saturation in said samples, whether a vitamin B12-related deficiency is due to a lack of secretion of intrinsic factor or to a malabsorption of intrinsic-factor-bound cobalamin in the intestine.

73. A method for evaluating whether a vitamin B12-related deficiency is due to a deficient transfer of cobalamin from food to intrinsic factor, said method comprising carrying out the method of claim 47, and further comprising the step of evaluating, on the basis of comparison of said concentration and/or saturation in said samples, whether the vitamin B12-related deficiency is due to a deficient transfer of cobalamin from food to intrinsic factor.

74. A method for diagnosing a vitamin B12 deficiency in an individual comprising the steps of:
i) obtaining a blood sample from an individual,
ii) having said individual ingest a dose of non-radioactive cobalamin or an analogue thereof, together with a binding protein or without a binding protein,
iii) obtaining, after a time period sufficient to allow uptake, if any, of the cobalamin or analogue thereof in the blood stream, a second blood sample from said individual,
iv) determining in said samples one or more selected from the group consisting of: the concentration of holo-TC, the concentration of holo-HC, the saturation of TC and the saturation of HC, and
v) determining, on the basis of comparison of said concentration and/or saturation in said two samples, whether said cobalamin or analogue thereof has been absorbed in the blood stream.

75. A kit-of-parts suitable for use in the diagnosis of a vitamin B12-related deficiency, comprising
i) materials suitable for determining the holoTC and/or holoHC concentration in a blood sample, and
ii) instructions to the user comprising a description of the possible use of the kit in carrying out the method defined in claim 42.

76. The kit-of-parts according to claim 75, wherein the materials for determining the holoTC and/or holoHC concentrations comprise antibodies to transcobalamin and/or antibodies to haptocorrin.

77. The kit-of-parts according to claim 75, further comprising non-radioactive cobalamin.

78. The kit-of-parts according to claim 75, further comprising intrinsic factor and/or haptocorrin.

79. The kit-of-parts according to claim 75, further comprising cobalarnin bound to a solid support and/or buffers, plastic material and substrates necessary determining the concentration of holo-TC and/or holo-HC.

80. The kit-of-parts according to claim 75, further comprising labelled cobalamin.
